(19) United States
(12) Patent Application Publication
(10) Pub. No.: US 2023/0117989 A1
(43) Pub. Date: Apr. 20, 2023

(54) ORGANIC AMINE PURIFICATION METHOD

(71) Applicants: Dow Global Technologies LLC, Midland, MI (US); Dow Chemical Korea Limited, Seoul (KP)

(72) Inventors: Qi Jiang, Shanghai (CN); Yujun Liu, Pearland, TX (US); Kaoru Ohba, Tokyo (JP); Jongcheol Kim, Seoul (KR); Xue Chen, Manvel, TX (US); Li Mu, Shanghai (CN); Jianhai Mu, Shanghai (CN); Stephen W. King, Braselton, GA (US)

(21) Appl. No.: 17/905,877

(22) PCT Filed: Apr. 8, 2020
(86) PCT No.: PCT/CN2020/083624

§ 371 Date: Sep. 8, 2022

(51) Int. Cl. C07C 213/10; C07C 215/06

(52) U.S. Cl. CPC C07C 213/10; C07C 215/06

(57) ABSTRACT

A method for purification of organic amines, comprising introducing a resin polymer matrix to a liquid containing at least an organic amine bonded to at least one metallic element, wherein the resin polymer matrix is embedded with an amino compound selected from the group consisting of iminodiacetic acid, aminomethylphosphonic acid or a combination thereof, and wherein the embedded resin polymer matrix binds the at least one metallic element, and the at least one metallic element is removed from the organic amine.

Organic amines are good ligands for metal ions and thus, metal impurities are a common issue when producing organic amines. Presently, there is no reliable method of removing metal impurities from organic amines. Methods that do exist for removal of metal impurities from aqueous and/or inorganic liquids leave significant metal ions in the treated liquid. One example of this is the use of chelation resins in treating aqueous and/or inorganic brines. The chelation resins are typically used to selectively remove transition metals or noble metals from these liquids and while common; leave a significant amount of metal in the treated liquid (e.g., an amount detectable at parts per million). Additionally, these processes are only suitable for the treatment of wastewater, inorganic brines, etc. with no such process for treating organic amines presently available.

For all these reasons and more, there is a need for method of purification of organic amines.

### SUMMARY

Embodiments relate to a method for purification of organic amines, comprising introducing a resin polymer matrix to a liquid containing at least an organic amine bonded to at least one metallic element, wherein the resin polymer matrix is embedded with an amino compound selected from the group consisting of iminodiacetic acid, aminomethylphosphonic acid or a combination thereof, and wherein the embedded resin polymer matrix binds the at least one metallic element, and the at least one metallic element is removed from the organic amine.

### DETAILED DESCRIPTION

The present disclosure relates to an organic amine purification process or method. This method entails the use of an ion exchange resin featuring iminodiacetic acid or aminomethylphosphonic acid (or both). Iminodiacetic acid, HN(CH□CO□OH)□, often abbreviated to IDA, is a dicarboxylic acid amine. The iminodiacetate anion can act as a tridentate ligand to form a complex with metal ions. Aminomethylphosphonic acid, CH₆NO₃P, abbreviated to (AMPA) is a weak organic acid with a phosphonic acid group which is capable of binding different metal ions mainly through oxygen atoms of the phosphonic acid group.

The ion exchange resin, in a preferred embodiment, may be described as a polymer matrix comprised of polyacrylate or polystyrene-divinylbenzene (or a mixture of the two). The IDA and/or AMPA is embedded within, throughout, and/or upon this polymer matrix. The IDA and/or AMPA may be introduced during formation of the polymer resin and this resin may be formed into beads resulting in the AMPA or IDA embedded inside the resin beads and on the surface. The AMPA or IDA may also be applied at a later step after the resin matrix is formed, resulting in a surface coating only. In a preferred embodiment, the concentration of AMPA or IDA in a resin ranges from 20 wt. % to 70 wt. % and more preferably from 40 wt. % to 60 wt. %. Generally, the higher concentration of AMPA or IDA utilized result in higher metal removal rate, however if the concentration is too high, the polymer matrix may become unstable.

The pore size of the polymer matrix may vary, with one embodiment having a preferred range from 1-2000 nm. This pore size is determined via ISO 9277:2010 the determination of the specific surface area of solids by gas adsorption (the BET method). The IDA/AMPA resin polymer matrix may be formed into beads, with the distribution of particle diameter ranging from 100-2,000μ. IDA and/or AMPA embedded resins can be mixed with each other at ratio 100:0 to 0:100. Consistent bead size may be obtained by use of a few meshes with different pore sizes to filter the uniform size of resin bead step by step

Additionally, anion ion exchange resins can also be mixed with the IDA and/or AMPA embedded chelation ion exchange resins. Two such anion ion exchange resins are Amberlite IRA98 (methanaminium N,N,N-trimethyl hydroxide) and Amberjet 90000H (quaternary ammonium). The anion ion exchange resin is introduced to release hydroxyl anion (OH—). This step is anion resin is optional and does not reduce metal removal. Some metals in organic amines exist in a complex form and require a chelating resin with stronger complexing strength. The additional anion resin does not and cannot directly capture the complex metals, but they may act as de-complexing agent. The mechanism for this de-complexing, known in the art, releases OH— to form metal hydroxide which can be easier to capture by chelating resins.

When purifying organic amines, the presently disclosed process may feature the use of at least one ion exchange column filled with iminodiacetic acid containing resin or an aminomethylphosphonic embedded resin beads. This column may be fluidly connected in line or parallel to another ion exchange column filled with the other material (that is, an aminomethylphosphonic embedded resin or an iminodiacetic acid containing resin, respectively. The organic amine containing liquid is passed through these columns, in one embodiment, at a flow rate of 1 to 30 bed volume (BV) per hour. When used together in series, either of these columns can be placed upstream of the other. Additionally, other column(s) may be loaded with anion ion exchange resin(s) and connected upstream or downstream of the IDA and/or AMPA ion exchange column(s), passing the organic amine containing liquid through the series of columns and producing extremely pure organic amines.

In another embodiment, simple mixing of the ion exchange resin(s) with the amine liquid may also be utilized to purify the organic amines. Once mixed, the resin(s) are allowed to react with the organic amines and remove metal from them. Then liquid is then filtered to separate the purified organic amines from the other components in the liquid.

The use of these ion exchange resins can efficiently remove most types of metal. Notably, the disclosed process removes Ca, Sr, Ba, Fe, Mn, Cu and Zn from organic amines which are particularly difficult to remove. Metal types may also include Li, Na, K, Mg, Al, Cr, Co, Ni, Ag, Cd, Pb, Sb, Sn, Ru, Rh and other types of metals utilized by electronic devices. The types of metal ions captured may yet also include Cs, Ga, Hg, Se, Te, Tl, V, U, Ti, Au, Hf, Ir, Pt, W, and any other metal ion which can form a bond with IDA and/or AMPA. The total metal removal rate is around 90%, with an iron removal rate at over 80%. The content of these metals can be reduced to less than 1 parts per million, to parts per billion (e.g., 100 parts per billion) and even parts per trillion levels of scarcity. This is a dramatic improvement over current purification techniques.

The organic amines which can be purified by use of this method include, but are not limited to highly concentrated (with less than 1% by weight water, preferably less than 0.1%) N-methylethanolamine or the similar chemical structures, such as monoethanolamine, diethanolamine, triethanolamine, isopropanolamine, diisopropanolamine, triisopropanolamin, N-methyldiethanolamine, aminoethyleneethanolamine, etc. These close to pure amines may also be mixed together. The optimum temperature at which the organic amines can be purified varies, in a preferred embodiment, from the freezing point of the liquid organic amine up to 70° C. In this same preferred embodiment (or another), the viscosity of the organic amines to be purified ranges from 10 cP to 100 cP (as measured by ASTM D7042), with a pH value of 0.1 mol/L aqueous solution ranging from 10-13 (as measured by ASTM E70).

### EXAMPLES

### Example 1

In this example, an organic amine; N-methylethanolamine was purified via the use of an iminodiacetic acid embedded resin (Puromet® MTS9300 sourced from Purolite) under a controlled test. Puromet® MTS9300 is a wastewater treatment. It is not currently recognized as a potential treatment for organic amines and there are large differences between wastewater treatment and organic amine treatment including number of metal types, metal concentration, metal form, pH value, liquid viscosity, compatibility, etc.

The Puromet® MTS9300 resin was converted to hydrogen form as part of this purification method. Another iminodiacetic resin was also tested (DS-22 sourced from Organo®) as were aminomethylphosphonic acid embedded resins (Puromet® MTS9500 (Purolite®) and DS-21 (Organo®)) and were all also converted to hydrogen form. Other resins were utilized as part of this test for comparison including Puromet® MTS9570 (Purolite®), Amberlite® IRC76 and Amberlite® IRA98 (Organo®), and Amberlite® UP252 and Amberjet® 9000 OH (DuPont®). Information regarding the resins utilized can also be found in Table 1 and Table 2 below.

TABLE 1
Iminodiacetic and Aminomethylphosphonic Resins

| | Puromet ® | Puromet ® | Organo ® | Organo ® |
|---|---|---|---|---|
| | MTS9300 | MTS9500 | DS-21 | DS-22 |
| Matrix | Macroporous type | | | |
| Matrix | Polystyrene-divinylbenzene | | | |
| polymer | | | | |
| Functional | Iminodiacetic | Aminomethylphosphonic | Aminomethylphosphonic | Iminodiacetic |
| group | —N(CH₂COOH)₂ | —NCH₂PO₃H₂ | —NHCH₂PO₃H₂ | —N(CH₂COOH)₂ |

TABLE 2
Other Ion-exchange Resins Used for Comparison

| | Puromet ® | Amberlite ® | Amberlite ® | Amberlite ® | Amberjet ® |
|---|---|---|---|---|---|
| | MTS9570 | IRC76 | IRA98 | UP252 | 90000H |
| Matrix | Macroporous type | | | | |
| Matrix polymer | Polystyrene-divinylbenzene | Polyacrylate | Polystyrene-divinylbenzene | Polystyrene-divinylbenzene | Polystyrene-divinylbenzene |
| Functional group | Sulfonic acid/phosphonic | Carboxylic acid | Methanaminium N,N,N- | Sulfonic acid | Quaternary ammonium |
| | acid | | trimethyl hydroxide | | |
| | —SO₃H/—NHCH₂PO₃H₂ | —CO₃H₂ | —N(CH₃)₂CH₂OH | —SO₃H | —N(CH₃)₃OH |

Each resin was tested by taking a volume of each (100 mL in dehydrated form) then flushing them with 1 L deionized water. The washed resins were then dried in a vacuum at 50° C. and 10 mmHg for 24 hr. Each dried resin was then charged to a Teflon column with an internal diameter of 50 mm and length of 150 mm. The organic amine (N-methylethanolamine) was then allowed to flow through the resin filled columns at rate of 2-10 BV/hr to enable resin water displacement. The flow rate conditions were adjusted as needed to purify the appropriate amount of organic amine (values shown in Table 3A). The organic amine (N-methylethanolamine) was allowed to flow through the filled columns for 15 minutes before a sample of the purified amine was taken in a 50 mL PFA bottle. This same test was run on the comparative resins with the relevant recipes and flow rates shown in Table 3B.

TABLE 3A
Resin recipe and flow rate conditions

| | Example No. | | | | | | |
|---|---|---|---|---|---|---|---|
| | 1 | 2 | 3 | 4 | 5 | 6 | 7 |
| Resin recipe | Puromet ® | Puromet ® | Puromet ® | Puromet ® | Puromet ® | Organo ® | Organo ® |
| | MTS9300 | MTS9500 | MTS9300/Puromet ® | MTS9300/Puromet ® | MTS9300/Puromet ® | DS-21 | DS-22 |
| | | | MTS9500 | MTS9500 | MTS9500 | | |
| | | | (1:1 volume) | (1:1 volume) | (1:1 volume) | | |
| Flow rate (BV/Hr) | 6 | 6 | 6 | 10 | 6 | 6 | 6 |
| Amine lot No. | 1 | 1 | 1 | 1 | 2 | 1 | 1 |

TABLE 3B
Comparison Resin Recipes and Flow Rate Conditions

| | Comparative example No. | | | |
|---|---|---|---|---|
| | 1 | 2 | 3 | 4 |
| Resin recipe | Puromet ® | Amberlite ® | Amberlite ® | Amberlite ® |
| | MTS9570 | IRC76/Amberlite ® | IRC76/Amberlite ® | UP252/Amberjet ® 9000OH |
| | | IRA98 | IRA98 | (1:1 volume) |
| | | (1:1 volume) | (1:1 volume) | |
| Flow rate | 6 | 6 | 6 | 2 |
| (BV/Hr) | | | | |
| Amine lot No. | 1 | 1 | 3 | 4 |

The concentrations of metals in the purified N-methylethanolamine samples were then analyzed by Inductively Coupled Plasma-mass spectrometry (ICP-MS). A standard methodology for these ICP-MS tests was utilized and conducted in triplicate. The results of the ICP-MS test can be found below in tables 4-8. It should be noted that the metal concentration and metal element ratio prior to purification vary by the lot of N-methylethanolamine utilized in each test. This same variation from lot to lot would be found in any other type of organic amine tested and the lot information can be found in tables 3A and 3B.

TABLE 4
Evaluation results

| | | | | | | | |
|---|---|---|---|---|---|---|---|
| Item | Original | Example 1 | Example 2 | Example 3 | Example 4 | Example 6 | Example 7 |

Metal content (ppb)

| | | | | | | | |
|---|---|---|---|---|---|---|---|
| Li | 0.38 | 0.03 | 0.01 | 0 | 0.01 | 0 | 0 |
| Na | 119 | 2.00 | 5.30 | 2.05 | 5.10 | 0.31 | 0.34 |
| Mg | 4.9 | 0 | 0 | 0.20 | 0.25 | 0 | 0.12 |
| Al | 5.1 | 2.90 | 0.90 | 0.34 | 0 | 0.23 | 0.65 |
| K | 10 | 3.00 | 0 | 0 | 0 | 0.07 | 0.34 |
| Ca | 92 | <3 | <3 | 10.6 | 5.50 | 22 | 3.24 |
| Cr | 0.51 | 0.80 | 0.49 | 0.43 | 0.37 | 0 | 0.48 |
| Mn | 6.92 | 2.30 | 3.80 | 4.16 | 6.30 | 1.30 | 5.10 |
| Fe | 137 | 17.0 | 9.7 | 8.9 | 21.3 | 0.60 | 12.3 |
| Co | <0.1 | 0 | 0 | 0 | 0.02 | 0 | 0.01 |
| Ni | 0.51 | 0 | 0 | 0 | 0 | 0 | 0.14 |
| Cu | 5.6 | 0 | 5.00 | 1.05 | 1.40 | 3.91 | 1.68 |
| Sr | 0.12 | 0.05 | 0.05 | 0.03 | 0 | 0.03 | 0.03 |
| Ru | 0 | 0 | 0 | 0 | 0 | 0 | 0 |
| Rh | 0.02 | 0 | 0 | 0 | 0 | 0 | <0.03 |
| Pd | 0.07 | 0.30 | 0 | 0 | 0 | 0 | <0.10 |
| Ag | 0.11 | 0.03 | 0.02 | 0.05 | 0 | 0.02 | 0.05 |
| Cd | 0.19 | 0.04 | 0.05 | 0.04 | 0.03 | 0 | <0.10 |
| Sn | 0.50 | 0 | 0 | 0.55 | 0.88 | 0 | 0.55 |
| Sb | 0.11 | 0.29 | 0.29 | 0.14 | 0.21 | 0.08 | 0.09 |
| Ba | <0.1 | 0.06 | 0.1 | 0 | 0 | 0 | 0.03 |
| Pb | 0.72 | 0 | 0 | 0 | 0 | 0 | 0.02 |
| Sum of metals | 384 | 31.8 | 28.7 | 28.5 | 41.37 | 28.6 | 25.4 |
| Removal rate | | 91.7% | 92.5% | 92.6% | 89.3% | 92.6% | 93.4% |
| Iron removal rate | | 87.6% | 92.9% | 93.5% | 84.5% | 99.6% | 91.0% |

TABLE 5
Evaluation results (Example 5)

| Item | Original | Example 5 |
|---|---|---|
| Li | 0.01 | 0 |
| Na | 9.7 | 0.67 |
| Mg | 0.59 | 0.16 |
| Al | 0.26 | 0.33 |
| K | 31.5 | 1.46 |
| Ca | 56 | 4.57 |
| Cr | 0.19 | 0.12 |
| Mn | 0.46 | 0.24 |
| Fe | 15.4 | 3.05 |
| Co | 0.01 | 0.01 |
| Ni | 0.36 | 0.28 |
| Cu | 7.16 | 0.81 |
| Zn | 14.1 | 3.55 |
| Sr | 0.03 | 0.02 |
| Ru | 0.01 | 0.01 |
| Rh | 0.01 | 0.02 |
| Pd | 0.05 | 0.14 |
| Ag | 0.02 | 0.12 |
| Cd | 0.05 | 0.03 |
| Sn | 0.14 | 0.38 |
| Sb | 0.08 | 0.05 |
| Ba | 0.02 | 0.02 |
| Pb | 0.08 | 0.05 |
| Sum of metals | 136 | 16.1 |
| Removal rate | | 88.2% |
| Iron removal rate | | 80.2% |

TABLE 6
Evaluation results (Comparative Examples 1 and 2)

| | | Comparative | Comparative |
|---|---|---|---|
| Item | Original | example 1 | example 2 |
| Li | 0.38 | 0.03 | 0 |
| Na | 119 | 18.3 | 2.89 |
| Mg | 4.9 | 1.00 | 0.10 |
| Al | 5.1 | 1.00 | 0.60 |
| K | 10 | 0 | 0.68 |
| Ca | 92 | 4.00 | 1.25 |
| Cr | 0.51 | 0.86 | 1.71 |
| Mn | 6.92 | 5.40 | 6.30 |
| Fe | 137 | 119 | 95 |
| Co | <0.1 | 0 | 0 |
| Ni | 0.51 | 0 | 0.00 |
| Cu | 5.6 | 6.00 | 4.22 |
| Sr | 0.12 | 0.05 | 0.05 |
| Ru | 0 | 0 | 0 |
| Rh | 0.02 | 0 | 0 |
| Pd | 0.07 | 0 | 0 |
| Ag | 0.11 | 0.00 | 0 |
| Cd | 0.19 | 0.06 | 0 |
| Sn | 0.50 | 0 | 0 |
| Sb | 0.11 | 0.08 | 6.28 |
| Ba | <0.1 | 1.01 | 0 |
| Pb | 0.72 | 0.10 | 0 |
| Sum of metals | 384 | 157 | 119 |
| Removal rate | | 59.1% | 68.9% |
| Iron removal rate | | 13.1% | 30.7% |

TABLE 7
Evaluation results (Comparative Example 3)

| | | Comparative |
|---|---|---|
| Item | Original | example 3 |
| Li | 0 | 0 |
| Na | 1.41 | 0.80 |
| Mg | 0.73 | 0.18 |
| Al | 0.60 | 0.47 |
| K | 37 | 1.06 |
| Ca | 7.20 | 14.7 |
| Cr | 0 | 1.14 |
| Mn | 0 | 0 |
| Fe | 1.29 | 3.18 |
| Co | 0 | 0 |
| Ni | 0.16 | 0 |
| Cu | 42 | 16.0 |
| Zn | 5.50 | 0.70 |
| Sr | 0 | 0.70 |
| Ru | 0 | 0 |
| Rh | 0 | 0 |
| Pd | 0 | 0 |
| Ag | 0 | 0 |
| Cd | 0 | 0 |
| Sb | 0 | 9.0 |
| Ba | 0 | 0 |
| Pb | 0 | 0 |
| Sum of metals | 96 | 48 |
| Removal rate | | 50.0% |
| Iron removal rate | | Increased |

TABLE 8
Evaluation result (Comparative Example 4)

| | | Comparative |
|---|---|---|
| Item | Original | example 4 |
| Li | 0.72 | 0 |
| Na | 356 | 40 |
| Mg | 220 | 24 |
| Al | 686 | 22 |
| K | 146 | 41 |
| Ca | 1789 | 483 |
| Cr | 5.2 | 1.30 |
| Mn | 257 | 25 |
| Fe | 136 | 84 |
| Co | 0.44 | 0.12 |
| Ni | 4.9 | 0.90 |
| Cu | 18 | 5.80 |
| Sr | 3.0 | 0.90 |
| Ag | 0.1 | 0 |
| Cd | 0 | 0 |
| Sn | 22 | 7.00 |
| Ba | 3.2 | 0.19 |
| Pb | 89 | 1.90 |
| Sum of metals | 4170 | 936 |
| Removal rate | | 77.5% |
| Iron removal rate | | 38.2% |

As shown, both iminodiacetic resin (Puromet® MTS9300) and aminomethylphosphonic resin (Puromet® MTS9500) or their mixture can efficiently remove various metals from N-methylethanolamine. The total metal removal rate is well above 90% for most of the embodiments tested. Iron, a notably difficult ion to remove, can be reduced by over 80% by the presently disclosed methods. The comparison chelation resins tested such as Puromet® MTS9570 only removed, at best, 77.5% of the total metal ions present in the organic amine and 38.2% of iron. Thus, the use of iminodiacetic resin and aminomethylphosphonic resin are a novel and effective means of purifying organic amines.

1. A method for purification of organic amines, comprising:
introducing a resin polymer matrix to a liquid containing at least an organic amine bonded to at least one metallic element, wherein
the resin polymer matrix is embedded with an amino compound selected from the group consisting of iminodiacetic acid, aminomethylphosphonic acid or a combination thereof,
and wherein the embedded resin polymer matrix binds the at least one metallic element, and
the at least one metallic element is removed from the organic amine.

2. The method of claim 1, wherein the resin polymer matrix comprises polyacrylate or polystyrene-divinylbenzene.

3. The method of claim 1, wherein the pore size of the resin polymer matrix ranges from 1-2,000 nm as determined by specific surface area of solids by gas adsorption.

4. The method of claim 1, wherein the resin polymer matrix is introduced to the organic amine containing liquid as resin beads, the particle diameter of said beads ranging in size from 100-2000 μm.

5. The method of claim 1, wherein an anion ion exchange resin is also introduced to the liquid.

6. The method of claim 1, wherein the temperature of the liquid ranges from freezing to 70° C.

7. The method of claim 1, wherein the flow rate of the liquid ranges from 1 to 30 BV/hr.

8. The method of claim 1, wherein more than 80% of the metallic elements are removed from a liquid containing at least an organic amine bonded to at least one metallic element.

9. The method of claim 1, wherein after introducing a resin polymer matrix to a liquid containing at least an organic amine bonded to at least one metallic element the concentration of metallic elements in the liquid containing at least an organic amine are less than 1 part per million.

10. The method of claim 1, wherein the organic amine includes highly concentrated monoethanolamine, diethanolamine, triethanolamine, isopropanolamine, diisopropanolamine, triisopropanolamin, N-methyldiethanolamine, or aminoethyleneethanolamine.
